SEQUENCE LISTING (5 sequences)

SEQ ID NO: 1 - DNA, 20 bp, Artificial Sequence
  Synthetic Construct
catcgtgatccatagtgagc

SEQ ID NO: 2 - DNA, 24 bp, Artificial Sequence
  Synthetic Construct
aacccacatctttatctctgtcat

SEQ ID NO: 3 - protein, 5 aa, Artificial Sequence
  Synthetic Construct
DFFVG

SEQ ID NO: 4 - DNA, 42 bp, Artificial Sequence
  Synthetic Construct
tgcacctttgtttgaacccacatcttctgcaaagaacaccaa

SEQ ID NO: 5 - DNA, 14 bp, Artificial Sequence
  Synthetic Contruct
tgcacctttgtttg

(19) United States
(12) Patent Application Publication
(10) Pub. No.: US 2009/0304676 A1
(43) Pub. Date: Dec. 10, 2009

(54) COMPOSITIONS AND METHODS FOR ENHANCING COGNITIVE FUNCTION

(75) Inventors: Vijaya B. Kumar, St. Louis, MO (US); William A. Banks, St. Louis, MO (US); John E. Morley, St. Louis, MO (US)

Correspondence Address: GLOBAL PATENT GROUP - EDI, 10411 Clayton Road, Suite 304, ST. LOUIS, MO, 63131, US

(73) Assignee: ST. LOUIS UNIVERSITY, St. Louis, MO (US)

(21) Appl. No.: 12/093,288

(22) PCT Filed: Nov. 10, 2006
(86) PCT No.: PCT/US06/60782

§ 371 Date: May 9, 2008

Related U.S. Application Data
(60) Provisional application No. 60/735,316, filed on Nov. 10, 2005.

(51) Int. Cl. A61K 39/395; A61K 38/17; A61K 31/7088; A61P 25/00

(52) U.S. Cl. 424/130.1; 514/12; 514/44 A

(57) ABSTRACT

The present application provides methods and compositions for improving cognitive function and enhancing mental performance in subjects, including those suffering from memory deficiencies, by regulating the activity of beta amyloid (Aβ) in the brain of such subjects.

The present invention claims benefit of priority to U.S. Provisional Application Ser. No. 60/735,316, filed Nov. 10, 2005, the entire contents of which are hereby incorporated by reference.

### BACKGROUND OF THE INVENTION

A. Field of the Invention

The invention generally relates to compositions and methods for enhancing memory. More particularly, it concerns compositions and methods of treating memory deficiency in patients suffering from dementia.

B. Related Art

Alzheimer's disease is widely believed to be due to an excess of amyloid-beta (Aβ) protein. This Aβ peptide has been shown to be amnestic in vivo (Flood et al., 1994a; Flood et al., 1991; Flood et al., 1994b; Terranova et al., 1996; Cleary et al., 1995). Transgenic mice which overproduce amyloid precursor protein have been shown to have a decreased memory (Hardy and Selkoe, 2002; Robinson and Bishop 2002). Despite a large literature on Aβ protein, its physiological role remains uncertain. Long term potentiation (LTP) is believed to be the synaptic correlate of memory formation. Most studies have shown that Aβ protein inhibits LTP (Itoh et al., 1999; Raymond et al., 2003), though few have shown that Aβ protein increases LTP (Wu et al., 1995; Kim et al., 2001; Trubetskaya et al., 2003). Further support for the concept that Aβ may stimulate LTP comes from amyloid precursor protein (APP) null mice, which have reduced synapses, impaired LTP, and which perform poor on spatial memory tasks (Dawson et al., 1990; Seabrook et al., 1999). Furthermore, presenilin-1-deficient mice have a reduced level of amyloid-beta peptide and impaired LTP (Morton et al., 2002). While antibodies against APP improve memory in the SAMP8 mouse, a strain which overproduce Aβ protein (Morley et al, 2002), they impair performance of passive avoidance tasks in rats (Huber et al., 1993) and chicks (Mileusnic et al., 2000). In the studies reported here, the inventors demonstrate that in young mice inhibition of APP with antibodies, inhibition of Aβ production with antisense to mRNA for APP or blocking the effect of amyloid-beta with a competitive receptor antagonist all impair learning. In addition, the inventors have shown that low doses of Aβ (12-28) enhances memory in young mice. While not wishing to be bound by theory, these data have lead the inventors to suggest that memory consolidation represents a physiological role for Aβ protein.

Aβ protein is well recognized in the art as having a significant role in the pathogenesis of Alzheimer's disease (AD). Overexpression of Aβ as in AD results impaired learning and memory. Reduction of Aβ in mouse models that overexpress Aβ, results in improvement of learning and memory. The presence of Aβ and its physiological role in non-disease states are not as clear. Recent studies have suggested that Aβ, which is secreted by neurons during excitatory neuronal activity downregulates excitatory synaptic transmission. This negative feedback loop suggests that Aβ provides a homeostatic mechanism to maintain appropriate levels of neuronal activity. If this is true, then not only would too much Aβ be a problem, but so would too little. The inventors sought to determine if endogenous AD has a role in learning and memory in young non-demented mice, which can reasonably be extrapolated to humans.

U.S. Pat. No. 6,310,048 and WO01/42266, which are herein incorporate by reference, and whose inventor is an inventor of the instant invention, discloses antisense molecules directed to portions of the APP (amyloid protein precursor) and/or Aβ message. The antisense molecules were shown to reduce the expression of APP and/or Aβ in cells transfected with the antisense molecules, and to increase the memory function of SAM-P8 mice in a foot shock avoidance test. The mice were administered the antisense molecules via intracerebral ventricular injection (i.c.v.). Nonetheless, improved reagents of this nature of still in great need.

### SUMMARY OF THE INVENTION

Surprisingly, the inventors have now shown that beta amyloid (Aβ) protein has a physiological role in learning and memory. They show that reducing the levels of Aβ protein in unimpaired mice results in impaired learning memory, and that administering low doses of Aβ protein to mice improves learning and memory. Thus, the invention is directed to compositions and methods for improving memory, treating memory deficiencies, and enhancing mental performance by regulating the activity of Aβ in the brain of a subject.

Thus, the invention is directed to compositions that modulate the cognition function of a vertebrate. Those compositions include agents that modulate the activity of Aβ protein in the brain or CNS of the vertebrate. A particular cognitive function is memory, as measured by the ability to perform an Acquisition of T-maze Footshock Avoidance test. A particular vertebrate is a mammal, such as a mouse, including a human. Agents include, but are not limited to drugs (e.g., DFFVG (SEQ ID NO:11 antibodies to Aβ or its precursor, amyloid precursor protein (APP) (e.g., monoclonal polyclonal, phage display), aptamers to Aβ/APP, polynucleotides to Aβ/APP (e.g., antisense RNA, protein nucleic acids (PNA), antisense DNA, RNAi, siRNA, and modifications thereof), and polypeptides. Polynucleotides include antisense RNAs that are complementary to the coding sequence for the C-terminal portion of Aβ/APP. Polypeptides include those corresponding to amino acids 12-28 of Aβ and amino acids 1-42 of Aβ. Any and all Aβ and APP polypeptides, as well as conservative substitutions thereof, may serve as the basis of these agents. Human and murine Aβ and APP polypeptide sequences are provided in the appended sequence listing.

In another embodiment, the invention is directed to methods for improving cognitive function is a subject, comprising administering to the subject one of more of the compositions and/or agents set forth above. Therapeutically effective amounts of the composition or agent may be administered in a pharmaceutically acceptable form to the subject via intravenous, intraparetoneal, subcutaneous, intracerebral ventricular injection (ICV), intrathecal, oral, inhalation, intranasal, and the like, administration. Methods of administration are by inhalation and by intranasal administration. Subjects are vertebrates and mammals, including humans.

The inventors have also made the surprising discovery that bivalent chimeral polynucleotides complementary to the sense sequence of a polynucleotide that encodes an Aβprotein and/or APP and another polypeptide known in the art to be involved in brain function, upon administration to a mammal having normal cognitive function, a dementia disease, Alzheimer's disease, neurofibrillary tangles and senile plaques, and/or the like, can improve the cognitive function of that mammal. Thus, the invention is further directed to compositions and methods useful in the improvement of cognitive function in patients suffering from dementia and/or in otherwise healthy subjects desiring to improve cognitive function. In U.S. Pat. No. 6,310,048, which is herein incorporated by reference, one of the instant inventors teaches the use of APP antisense molecules in the improvement of memory in a murine dementia model. The instant invention discloses improved polynucleotides that enhance memory function in vertebrates.

Thus, in other embodiments, the invention is directed to compositions and methods useful in the improvement of the cognitive function of subjects, which may or may not suffer from a cognitive disorder, such as Alzheimer's disease. In one such embodiment, the composition comprises or consists of hybrid polynucleotides, which comprise a sequence complementary to an APP or Aβ sequence and another polypeptide useful to brain function. Compositions are directed to hybrids comprising an APP/Aβ antisense and a β-secretase (BACE) antisense sequence or presenilin-1 antisense sequence. More particular hybrid antisense sequences include the sequence as set forth in SEQ ID NO:9 (FIG. 6), SEQ ID NO:10 (FIG. 8) and equivalents thereof.

In yet the other embodiment, the method for improving cognitive function comprises the step of administering to a subject a therapeutically effective amount of a composition comprising a hybrid polynucleotide as set forth above (e.g., SEQ ID NOS:9 and 10). Therapeutically effective amounts of the composition may be administered in a pharmaceutically acceptable mixture to the subject via intravenous intraparetoneal, subcutaneous, intracerebral ventricular injection (ICV), oral, inhalation, intranasal, and the like administration. Methods of administration are by inhalation and by intranasal administration. Subjects are vertebrates, more particularly mammals, including humans.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1 depicts the effect of antibody to amyloid beta protein (Aβ) administered 72 hours prior to training of mice on Acquisition of T-maze Footshock Avoidance (ATMFA).

FIG. 2 depicts the effect of DFFVG (SEQ ID NO:11) administered 72 hours prior to training of mice on ATMFA.

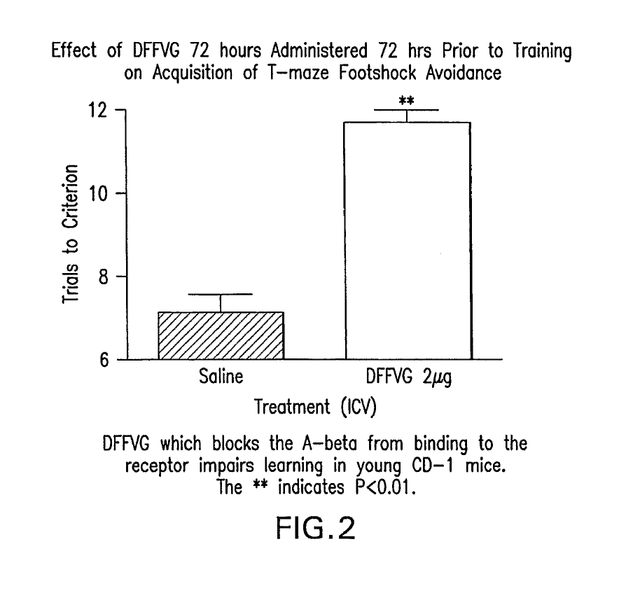

FIG. 3 depicts the effect of C-terminal antisense to Aβ administered three times prior to training of mice on ATMFA.

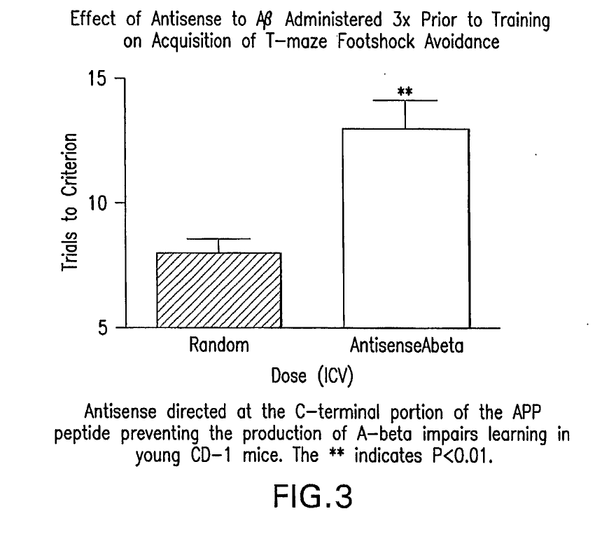

FIG. 4 depicts the effect of the peptide corresponding to Aβ amino acids 12-28 on the retention of the T-maze Footshock Avoidance test.

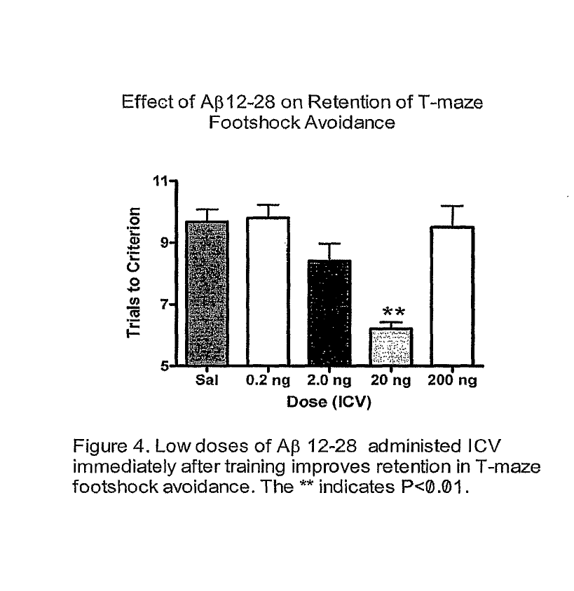

FIG. 5 depicts the effect of the peptide corresponding to amino acids 12-28 amino acids 1-48 on the retention of the T-maze Footshock Avoidance test.

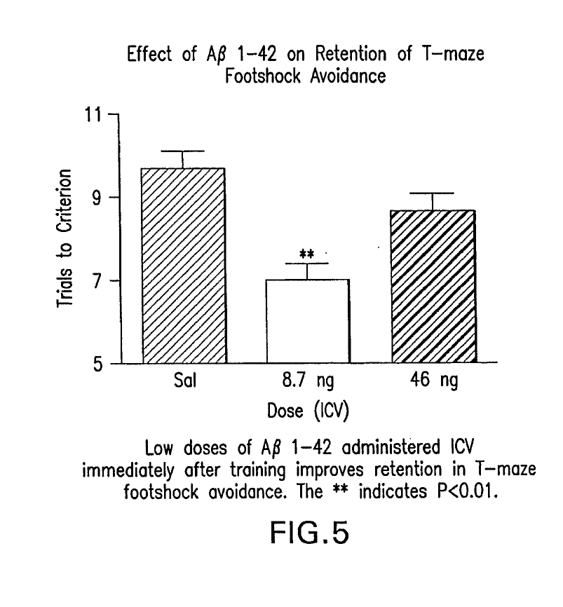

FIG. 6 depicts an exemplary hybrid (bivalent) antisense molecule that that targets APP and BACE (SEQ ID NO:9).

FIG. 7 depicts the quantified effects of an Aβ/BACE hybrid antisense in alzheimers model mice on the acquisition and retention in the T-maze Footshock Avoidance test.

FIG. 8 depicts an exemplary hybrid (bivalent) antisense molecule that targets APP and presenilin-1 (APP-PS1) (SEQ ID NO:10).

FIG. 9 depicts Western blots showing the downregulation of both APP and presenilin-1 by administration of an APP-presenilin hybrid antisense.

FIG. 10 demonstrates the stability of antisense oligonucleotides. Antisense oligonucleotides were labeled with γ-³²P-ATP and administered orally as described in the text. Four hours after administration the animal was sacrificed and specified tissues were processed. Soluble fractions of the tissues were subjected electrophoresis on 5% polyacrylamide gels as described in the text. Arrow indicates the position of major band. Star indicates the position of free oligonucleotide. Panel A- short antisense oligonucleotide (10 mer); panel B-long antisense oligonucleotide (42 met). Notice the streaking of radioactivity in some lanes suggesting the degradation.

FIG. 11 demonstrates the mobility of oligonucleotide after phenol-chloroform extraction. The tissue samples described in FIG. 10 which received a 42 mer antisense oligonucleotide were subjected to Phenol:chloroform (1:1) extraction followed by ethanol precipitation and subjected to electrophoresis on a 5% polyacrylamide gel. Arrow indicates the position of free oligonucleotide.

### DESCRIPTION OF DETAILED EMBODIMENTS OF THE INVENTION

As used herein, the terms “modulation” and “regulation” are meant to included either inhibition or stimulation of gene expression. In the context of the present invention, inhibition or stimulation of Aβ (and/or other brain important proteins) expression is the type of modulation that is desired. This modulation can be measured by methods that are known in the art and which are described in the examples that follow. Such methods have been described, among others, by Sambrook et al. (1989 ) For example, RNA expression can be measured by Northern blot or slot blot hybridization assays, primer extension and poly-A+ assays. Western blot assays, radioimmunoassays and enzyme-linked immunoabsorbent assays can be used to measure protein.

As used herein to describe the regulatory effect that a compound has on the expression of a gene, the term “inhibition” means that the compound reduces the expression of one or more genes to some degree compared with its expression under the same conditions, but without the presence of the compound. Inhibitory compounds commonly demonstrate concentration dependant activity, wherein increased concentrations of such compounds demonstrate higher levels of inhibition. When the terms “inhibitory effective amount” are used herein with respect to an inhibitory compound, what is meant is an amount of an antisense compound that inhibits the expression of a gene to a measurable degree. Such inhibitory effective amount preferably reduces the level of expression by at least about 25%; more preferably, by at least about 50%; even more preferably by at least about 75%; and most preferably by at least about 80%, or more.

The term “hybridization,” as used herein, means hydrogen bonding, which may be Watson-Crick, Hoogsteen or reversed Hoogsteen hydrogen bonding, between complementary nucleotide bases. For example, adenine and thiamine, and guanine and cytosine, respectively, are complementary nucleobases that pair through the formation of hydrogen bonds. “Complementary,” as that term is used herein, refers to the capacity for precise pairing between two nucleotides. For example, if a nucleotide at a certain position of an polynucleotide is capable of hydrogen bonding with a nucleotide at the same position of a DNA or RNA molecule, then the polynucleotide and the DNA or RNA are complementary to each other at that position. The polynucleotide and the DNA or RNA are complementary to each other when a sufficient number of corresponding positions in each molecule are occupied by nucleotides that can hydrogen bond with each other. “Specifically hybridize” means that a particular sequence has a sufficient degree of complementarity or precise pairing with a DNA or RNA target sequence that stable and specific binding occurs between the polynucleotide and the DNA or RNA target. It is understood in the art that the sequence of an antisense compound need not be 100% complementary to that of its target nucleic acid to be specifically hybridizable. Typically, for specific hybridization in vitro, moderate stringency conditions are used such that hybridization occurs between substantially similar nucleic acids, but not between dissimilar nuleic acids. In in vitro systems, stringency conditions are dependent upon time, temperature and salt concentration as can be readily determined by the skilled artisan (see, e.g., Sambrook et al., 1989). For in vivo antisense methods, the hybridization conditions consist of intracellular conditions which govern the hybridization of the antisense polynucleotide with the target sequence. An antisense compound specifically hybridizes to the target sequence when binding of the compound to the target DNA or RNA molecule interferes with the normal translation of the target DNA or RNA such that a functional gene product is not produced, and there is a sufficient degree of complementarity to avoid non-specific binding.

As used herein, the term “antisense molecule” is meant to include, but not be limited to, antisense polynucleotides, and is intended to include other chemical compounds that specifically bind to the same targeted nucleic acids that are described below, and that provide the same regulatory effect on AD (and/or other important brain protein) expression as the subject antisense polynucleotides. The antisense polynucleotides of the present invention are synthesized in vitro and do not include antisense polynucleotides of biological origin, except for polynucleotides that comprise the subject antisense polynucleotides and which have been purified from or isolated from such biological material. Particular antisense polynucleotides of the present invention are: CATCGTGATCC-ATAGTGAGC; (SEQ ID NO: 9); and/or AACCCACATCTTTATCTCTGTCAT; (SEQ ID NO:10).

While antisense polynucleotides are one form of antisense compound, the present invention contemplates other oligomeric antisense compounds, including, but not limited to, polynucleotide mimetics those containing modified backbones (which may be referred to herein as “modified internucleoside linkages”), and/or 3′ and 5′ terminal moieties that provide physiological or other stability. As defined herein, polynucleotides having modified backbones include those that retain a phosphorous atom in the backbone, as well as those that do not have a phosphorous atom in the backbone.

Modified polynucleotide backbones which are useful in the subject antisense polynucleotides include, for example, phosphorothioates, chiral phosphorothioates, phosphorodithioates, phosphotriesters, aminoalkylkphosphotriesters, methyl and other alkyl phosphonates including 3′-alkylene phosphonates and chiral phosphonates phosphinates, phosphoramidates including 3′-amino phosphoramidate and aminoalkylphosphoramidates, thionophosphoramidates, thionoalkylphosphonates, and boranophosphonates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid form are also included. References that teach the preparation of such modified backbone polynucleotides are provided, for example, in U.S. Pat. No. 5,945,290, incorporate by reference.

Modified polynucleotide backbones that do not include a phosphorous atom therein may comprise short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; alkene-containing backbones; sulfamate backbones; methyleneimino and methylenehydrazino backbones; sulfonate and sulfonamide backbones; amide backbones; and others having mixed N, O, S and CH₂ component parts. Preparation of the polynucleotides listed above is described in U.S. Pat. No. 5,945,290.

Other useful polynucleotide mimetics, which are useful in the subject antisense polynucleotides, comprise replacement of both the sugar and the internucleoside linkage, i.e., the backbone, of the nucleotide units with novel groups. One such oligomeric compound that has excellent hybridization properties is a peptide nucleic acid. See, e.g., Nielsen et al, (1991); and U.S. Pat. Nos. 5,539,082; 5,714,331; and 5,719,262, incorporated by reference. In such peptide nucleic acid compounds, the sugar-backbone of an polynucleotide is replaced with an amide containing backbone, in particular with an aminoethylglycine backbone. The nucleobases are retained and are bound directly or indirectly to aza-nitrogen atoms of the amide portion of the backbone.

Other useful modified polynucleotides are those having phosphorothioate backbones and polynucleotides with heteroatom backbones, and in particular —Ch₂—NH—O—CH₂—, —CH₂—N(CH₃)—O—CH₂—, —CH₂—O—N(CH₃)—CH₂—, —CH₂—N(CH₃)—N(CH₃)—CH₂—, and —O—N(CH₃)—CH₂—CH₂—, wherein the native phosphodiester backbone is represented as —O—P—O—CH₂—, as disclosed in U.S. U.S. Pat. No. 5,489,677, and the amide backbones disclosed in U.S. Pat. No. 5,602,240. Also useful are polynucleotides having morpholino backbone structures as taught in U.S. U.S. Pat. No. 5,304,506. Each of the preceding patents is incorporated by reference.

Modified polynucleotides can also contain one or more substituted sugar moieties (which may be referred to herein as “modified sugar moieties”). Useful polynucleotides comprise one of the following at the 2′ position: OH; F; O—, S—, N-alkyl; N-alkenyl; N-alkynyl; or O-alkyl-O-alkyl, wherein the alkyl, alkenyl, or alkynyl may be substituted or unsubstituted C₁ to C₁₀ alkyl, or C₂ to C₁₀ alkenyl and alkynyl; O(CH₂)O(CH₃); O(CH₂)O(CH₂)ₙCH₃; O(CH₂)ₙNH₂; or O(CH₂)ₙCH₃ (where n=1 to 10); Cl; Br; CNB; CF₃; OCF₃; NO₂; N₃; NH₂; heterocycloalkyl heterocycloalkaryl; aminoalkylamino; polyalkylamino; substituted silyl; an RNA cleaving group; a cholesterol group; a reporter group; an intercalator; a group for improving the pharmacokinetic properties of an polynucleotide; or a group for improving other substituents having similar properties. Polynucleotides can also have sugar mimetics such as cyclobutyls in place of the pentafuranosyl group. A particular modified sugar moiety is a 2′-O-methoxyethyl sugar moiety.

Other useful antisense compounds may include at least one nucleobase modification or substitution. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases, such as 5-methylcytosine, 5-hydroxymethylcytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocystine, 5-halouracil and cytosine, 5-propynyl uracil and cytosine, 6-azo uracil, cytosine and thymine, 5-uracil, 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl and other 5-substitutes uracils and cytosines, 7-methylguanine and 7-methyladenine, 8-azaguanine and 8-azaadenine, 7-deazaguanine and 7-deazaadenine and 3-deazaguanine and 3-deazaadenine.

The antisense compounds of the present invention may be conveniently and routinely made through the well-known technique of solid phase synthesis. Equipment for such synthesis is available from several manufacturers and vendors including, for example, Applied Biosystems, Foster City, Calif. Any other means for such synthesis known in the art may additionally or alternatively be employed. It is also well known to use similar techniques to prepare modified polynucleotides such as the phosphorothionates and alkylated derivatives that are discussed above.

The subject antisense compounds can be used in a method to modulate the expression of Aβ and/or other brain important proteins (e.g., presenilin 1 and BACA) in cells or tissues. In the method, one or more of the antisense compounds, is contacted with the cells or tissues. In a particular embodiment, the antisense compound(s) is administered in an Aβ et alia inhibitory effective amount. The effect of the antisense compound(s) is to inhibit the expression of APP by the cells or tissues.

For therapeutics, methods of treating a condition arising from abnormal Aβ expression and/or clearing are provided. As used herein, the terms “abnormal Aβ expression” refer to overproduction of Aβ, or production of mutant Aβ including, but not limited to, mutant forms of Aβ with mutations at codons 717, 670 and/or 671 (of APP770). The formulation of therapeutic compositions and their subsequent administration is believed to be within the skill of those in the art. In general, for therapeutics, a patient suspected of having, or being prone to a disease or condition associated with the expression of Aβ can be treated by administering to the patient one or more of the subject antisense polynucleotides, commonly in a pharmaceutically acceptable carrier, in amounts and for periods of time which will vary depending upon the nature of the particular disease, its severity and the patient's overall condition. In general, the antisense compound is administered to the patient in an Aβ inhibitory effective amount. In one embodiment of the present invention, the disease is Alzheimer's disease. The subject may be a vertebrate, more particularly a mammal, and in particular a human.

A “pharmaceutically acceptable carrier” (also referred to herein as an “excipient”) is a pharmaceutically acceptable solvent, suspending agent or any other pharmacologically inert vehicle for delivering one or more of the subject antisense polynucleotides to an vertebrate. The pharmaceutically acceptable carrier may be a liquid or a solid and is selected with the planned manner of administration in mind so as to provide for the desired bulk, consistency, and other pertinent transport and chemical properties, when combined with one or more of the subject antisense polynucleotides and any other components of a given pharmaceutical composition. Typical pharmaceutically acceptable carriers include, but are not limited to, saline solution; binding agents (e.g., pregelatinized corn starch, polyvinylpyrrolidone or hydroxypropyl methylcellulose, or etc.); fillers (e.g., lactose and other sugars, microcrystalline cellulose, pectin, gelatin, calcium sulfate, ethyl cellulose, polyacrylates or calcium hydrogen phosphate, and the like); lubricants (e.g., magnesium stearate, starch, polyethylene glycol, sodium benzoate, sodium acetate, and the like); disintegrates (e.g., starch, sodium starch glycolate, and the like); or wetting agents (e.g., sodium lauryl sulfate, and the like).

The pharmaceutical compositions of this invention may be administered in a number of ways depending upon whether local or systemic treatment is desired, and upon the area to be treated. Administration may be topical (including opthalmic, vaginal, rectal, intranasal, transdermal), oral, nasal, bronchial or parenteral, for example, by intravenous drip, subcutaneous, intraperitoneal or intramuscular injection or intrathecal or intraventricular administration, such as, for example, by intracerebral ventricular injection (ICV), intrathecal intranasally, or by inhalation. It is believed that the subject antisense polynucleotides can also be administered by tablet, since the toxicity of the polynucleotides is very low. Administration can be either rapid as by injection or over a period of time as by slow infusion or administration of slow release formulations. For treating tissues in the central nervous system, administration can be by injection or infusion into the cerebrospinal fluid. When it is intended that the antisense polynucleotide of the present invention be administered to cells in the central nervous system, administration can be with one or more agents capable of promoting penetration of the subject antisense polynucleotide across the blood-brain barrier.

The subject antisense polynucleotides can also be linked or conjugated with agents that provide desirable pharmaceutical or pharmacodynamic properties. For example, the antisense polynucleotide can be coupled to any substance known in the art to promote penetration or transport across the blood-brain barrier such as an antibody to the transferrin receptor or other means of crossing the blood brain barrier (e.g., PACAP), and administered by intravenous or CSF injection. The antisense compound can be linked with a viral or other vector, for example, which can make the antisense compound more effective and/or increase the transport of the antisense compound across the blood-brain barrier.

The subject antisense compounds may be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. For example, cationic lipids may be included in the formulation to facilitate polynucleotide uptake. One such composition shown to facilitate uptake is LIPOFECTIN™ (available from GIBCO-BRL, Bethesda, Md.).

The antisense compounds of the present invention can include pharmaceutically acceptable salts, esters, or salts of such esters, or any other compound which, upon administration to an animal, including a human, is capable of providing, directly or indirectly, the biologically active metabolite or residue thereof. Accordingly, for example, the invention is also meant to include prodrugs and pharmaceutically acceptable salts of the compounds of the invention, pharmaceutically acceptable salts of such prodrugs, and other bioequivalents.

As used herein, the term “prodrug” means a therapeutic agent that is prepared in an inactive form that is converted to an active form within the body or cells thereof by the action of endogenous enzymes or other chemicals and/or conditions. The term “pharmaceutically acceptable salts” means physiologically and pharmaceutically acceptable salts of the compounds of the invention, i.e., salts that retain the desired biological activity of the parent compound and do not impart undesired toxicological effects thereto.

The present invention also includes pharmaceutical compositions and formulations that include the antisense compounds of the invention. Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for intrathecal or intraventricular administrations may include sterile aqueous solutions which may also contain buffers, diluents and other suitable additives.

Compositions for oral administration include powders or granules, suspensions or solutions in water or non-aqueous media, capsules, sachets or tablets. Thickeners, flavorings, diluents, emulsifiers, dispensing aids or binders may be desirable.

Formulations for parenteral administration may include sterile aqueous solutions which may also contain buffers, diluents and other suitable additives.

### EXAMPLES

The following examples describe particular embodiments of the invention. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples. In the examples, all percentages are given on a weight basis unless otherwise indicated.

### Example 1

### Aβ Peptides Enhance Memory Function in Mammals and Down-Regulators of Aβ Reduce Memory Function in Otherwise Healthy Mammals

SUMMARY: The inventors examined several ways of blocking endogenous Aβ in healthy 2 month old CD-1 mice. In a first study, mice were injected with 2 μl of the antibody to Aβ or Antibody to IgG as a control intracerebroventricularly (ICV) 72 hrs prior to training. Mice were trained in T-maze footshock avoidance. Antibody to Aβ impaired acquisition of T-maze footshock avoidance. In second study, the inventors blocked Aβ with a peptide, which blocks the amino acid sites 18-22 on Aβ peptide inactivating a crucial site for learning and memory on the peptide. When administered 72 hours prior to acquisition, the compound DFFVG (SEQ ID NO:11) impaired acquisition of T-maze footshock avoidance. The current results indicate that, in normal healthy young animals, the presence of Aβ is important for learning.

MICE: The subjects for the experiments were 8 week old CD-1 male mice obtained from a breeding colony. The mice are tested regularly to ensure they are virus and pathogen free. Food (Lab Diet 5001 Rodent Diet, PMI Nutrition International LLC, Brentwood, Mo.) and water were available on an ad lib basis and the rooms had a 12 hour light-dark cycle with lights on at 0600 hours.

DRUGS: Beta amyloid 1-42 (Aβ) was purchased from American Peptide Co. (Sunnyvale, Calif.). Aβ 12-28 was purchased from Phoenix Pharmaceuticals, Inc. (Belmont Calif.). Antisense polynucleotide was purchased from Midland Certified Reagent Co. (Midland, Tex.). Antibody to Aβ was purchased from DAKO Corporation (Carpinteria, Calif.).

TRAINING AND TESTING: Mice were trained in T-maze footshock avoidance. In the T-maze, mice were trained until they made one avoidance. One week later retention was tested by continuing training until mice made 5 avoidances in 6 consecutive trials.

SURGERY AND DRUG ADMINISTRATION: Forty-eight hours prior to testing, the mice were prepared for ICV injection. A unilateral hole was drilled 0.5 mm posterior to and 1.0 mm to the right of the bregma. The injection depth was 2.0 mm into the third ventricle. Immediately after training, the mice were again placed under light anesthesia, and injected ICV with 2.0 μl of Triolein, Olein or saline. The injection was delivered over 30 seconds through a 30 gauge needle, which was attached to a 10 μl syringe. After ICV injection, the scalp was closed and the mice were returned to their cages.

STATISTICS: Results were expressed as means with their standard errors. The retention test scores were analyzed by one-way analysis of variance (ANOVA) for each group followed by Dunnett's t-test post hoc analysis.

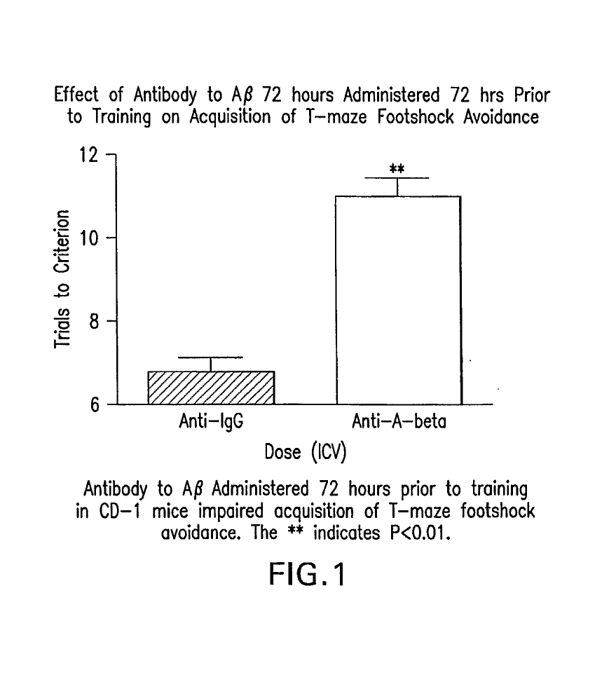

RESULTS: Anti-Aβ antibodies were delivered to the brain ventricle of healthy mice (supra) 72 hours prior to a T-maze footshock avoidance trial. Control animals were given anti-IgG antibodies. As is shown in FIG. 1, the average number of trials to attain the criterion for animals receiving the amyloid beta protein inhibitor was increased more than 50% over the average number of trials for control animals. This result suggests that inhibitors (down regulators) of Aβ, when delivered to the brains of an animal, such as a mammal, can impair cognitive function such as memory.

DFFVG (SEQ ID NO:11) was delivered to the brain ventricle of healthy mice (supra) 72 hours prior to a T-maze footshock avoidance trial. Control animals were given anti-IgG antibodies. As is shown in FIG. 1, the average number of trials to attain the criterion for animals receiving the amyloid beta protein inhibitor was increased more than 70% over the average number of trials for control animals. This result suggests that inhibitors (down regulators) of Aβ, when delivered to the brains of an animal, such as a mammal, can impair cognitive function such as memory.

### Example 2

### Bivalent (Hybrid) Antisense Molecules Modulate Cognitive Function

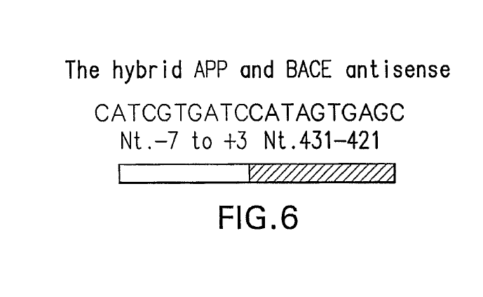

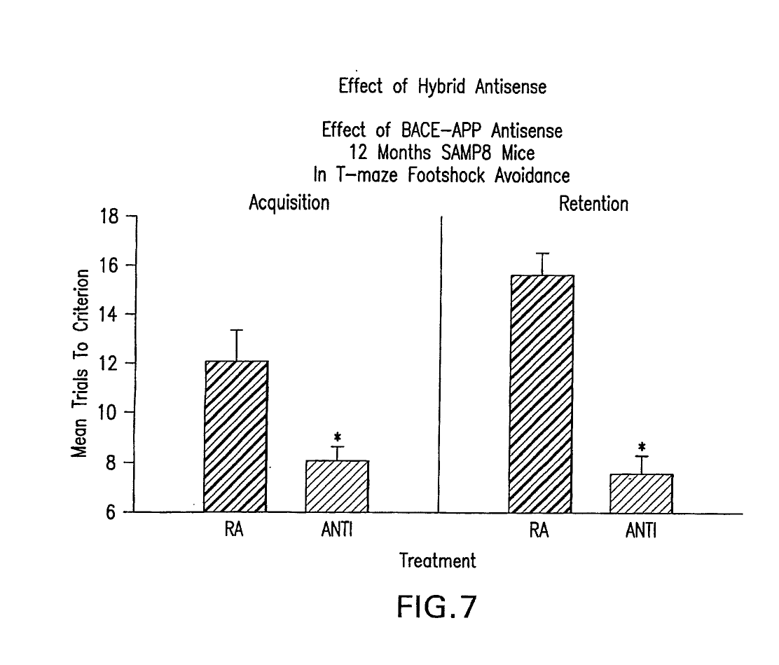

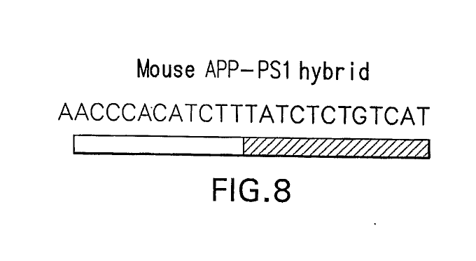

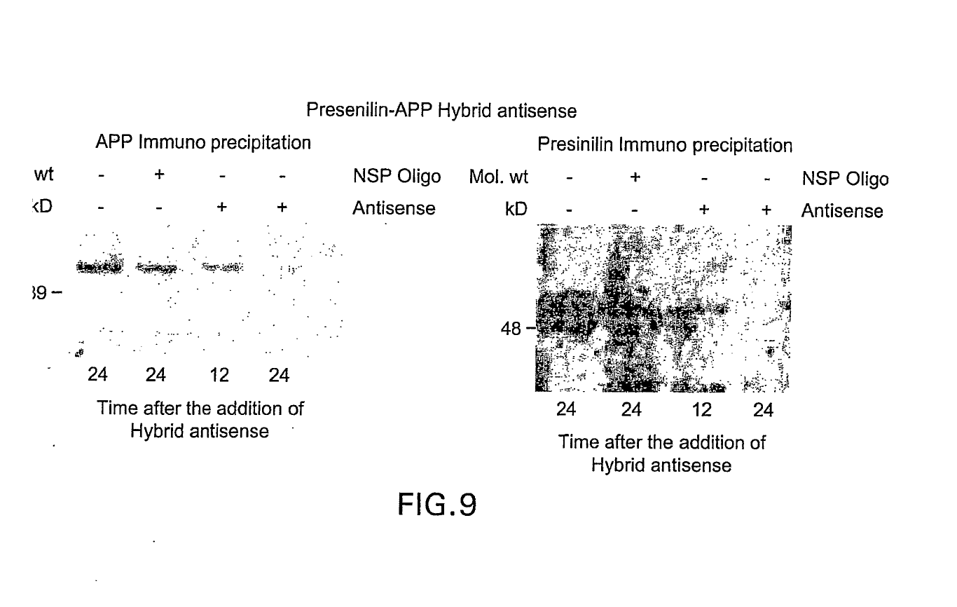

FIGS. 6-9 depict experiments that demonstrate the effectiveness of hybrid antisense molecules to reducing the level of Aβ, APP, presenilin-1, and BACE. The inventors envision that these and other equivalent hybrid antisense molecules can be effective in enhancing memory in subjects.

### Example 3

### Orally Administered Antisense Oligonucleotides Cross Blood Brain Barrier in Mice

INTRODUCTION: The use of complimentary sequences to arrest translation of mRNAs was described in the late 1970's (Paterson et al., 1977; Hastie and Held, 1978; Zamecnik and Stephenson, 1978). However, use of antisense oligonucleotides for selective blockage of specific mRNAs and elucidation of the mechanisms of antisense inhibition in cells is of recent origin (Bennet et al., 1985; Milligan et al., 1993; Wagner, 1994; Walder and Walder, 1988; Crooke, 1993; Dolnick, 1991). The decrease in mRNA levels mediated by oligonucleotides at concentrations similar to those used in this investigation has been reported for several proteins (Walder and Walder, 1988; Bennett et al., 1994; Chiang et al., 1991). Cellular assays have indicated that antisense oligonucleotides containing phoshodiester or thionated linkages act through RNase H cleavage of 2′-deoxy oligonucleotides (Chiang et al., 1991; Walder and Walder, 1988; Wagner et al., 1993). In certain cases, RNase H-independent inhibition by antisense oligonucleotide targets the translation initiation codon or 5′ untranslated region of RNA (Johansson et al., 1994). Irrespective of mechanism, the effectiveness of antisense oligonucleotide depends on its efficient transport to the specific site of action. It has recently been shown that i.c.v. injection of antisense oligodeoxynucleotides protected by 2′-O-methoxy ethyl ribosyl modification were rapidly taken up. Labeled antisense ODNs were observed to penetrate across the cell membrane and accumulate in both nuclear and cytoplasmic compartments of neuronal cells in mouse brain (Chauhan, 2002). Uptake in an in vitro cell system is shown to occur by a simple diffusion through the pores generated during cell manipulation. In vivo transport appears to take place by as yet unknown mechanisms. Tissue distribution essentially determines the amount of oligonucleotide needed to administer to successfully blockade the targeted message in a specific tissue. The inventors have previously shown that phosphorothiated antisense molecules are effective in brain after direct injection into the brain or after intravenous administration. Whether uptake occurs after oral administration is important drug delivery issue. Here, they investigated the tissue distribution of antisense oligonucleotides of two different sizes by oral administration in c57BL/6 mice.

LABELING OF OLIGONUCLEOTIDES: Two oligonucleotides complementary to Amyloid Precursor Protein (APP) mRNA (5′-TGCACCTTTGTTTGAACCCACATCTTCTG-CAAAGAACACCAA-42mer) (SEQ ID NO. 12) and (5′-TGCACCTTTGTTTG-10 mer) (SEQ ID NO:13) were synthesized by Midland Certified Reagent company, Midland Tex. For greater stability, the nucleotides were phosphorothiated. 5′-endlabeling with γ-³²P labeled ATP (NEN, MA) was performed as described by us earlier (Banks et al., 2001). Briefly 2-3 μg of oligonucleotide in 70 mM Tris-HCl (pH 7.6) containing 10 mM MgCl₂, 5 mM dithothreotol and 100 μCi of γ-³²P labeled ATP was incubated at 37° C. for 1 hr with 10 units of T₄ polynucleotide kinase in a total volume of 15 μl. At the end of the reaction the kinase was inactivated by heating the sample to 68° C. in a water bath. Labeled oligonucleotide was separated from unreacted radioactivity by alcohol precipitation. Purity was ascertained by polyacrylamide gel electrophoresis. Generally the specific activity of a labeled oligonucleotide was ˜100×10⁶ cmp/μg.

ADMINISTRATION OF RADIOLABELED OLIGONUCLEOTIDES: For oral administration of oligonucleotides, a tygon tubing with a small metal ball was inserted into the gut of the 4 month old C57BL/6 mice (obtained from National Institute of Aging, Bethesda Md.). Radiolabeled oligonucleotide in 200 μl volume was pumped in to the stomach with a 1 ml tuberculin syringe attached to the tygon tubing.

SAMPLE PREPARATION: Various tissues from the mouse were harvested 1 and 4 hours after administration of labeled oligonucleotides. Precisely weighed quantities of each tissue are homogenized in 10 mM Tris-HCl, 2 mM EDTA. The homogenate was centrifuged at 14,000×g for 5 min in a microfuge at 4° C. The supernatant was heated to 95° C. in a water bath and the denatured protein was removed by a 14,000×g centrifugation at 4° C. Counts/minute (cpm) of radioactivity was measured in the pellets and supernatant and results expressed as percent (as determined by the following formula) distribution in each tissue.

$$\{ \frac{cpm obtained in the supernatant + cpm obtained in the pellet}{wt . of the tissue} \} \times \{ \frac{100}{cpm administered} \}$$

STABILITY OF OLIGONUCLEOTIDES: The supernatant sample obtained by tissue homogenization as described above was lyophilized and solublized in 50 μl of TBE (Tris-Borate-EDTA) buffer, pH 8.3. The solution was briefly centrifuged in a microfuge to remove undissolved material and applied to either 5% or 12 polyacrylamide gels in the presence of bromophenol blue and xylene cyanol as the marker dyes. The samples were subjected to electrophoresis until bromophenol blue was 12 cm from the origin. At the end of the electrophoresis, gels were dried and exposed to X-Omat X-ray film (Eastman Kodak, Rochester N.Y.) for required lengths of time.

ORAL ADMINISTRATION OF OLIGONUCLEOTIDES: A typical distribution of radioactive short and long oligonucleotides one hour after oral administration is shown in Table 1. The results show the distribution of short and long oligonucleotides in spleen, liver, brain, kidney, pancreas and blood. In the case of short oligonucleotide, major amount 1.12±0.13%/gm was present in spleen followed by liver 0.37±0.028%/gm and kidney 0.289±0.026%/gm one hour after administration. Pancreas showed 0.079±0.042% of the administered oligonucleotide. About 0.0074±0.002%/gm was recovered in the brain. This is in conformity with earlier observations that delivery of the oligonucleotide directly into the blood by intravenous (i.v.) injection resulted in the appearance of oligonucleotide in the brain in a short time (Banks et al., 2001). The overall pattern of tissue distribution was the same for longer (42-mer) antisense oligonucleotide. Major amount from blood appears to go to spleen in the initial stages after administration. Further, the size of 10 or 42 nucleotides in length did not have noticeable difference in the in tissue distribution (Table 1).

TABLE 1
Distribution and recovery of antisense oligonucleotides in various tissues one hour after administration.

| Size of | | | | | | |
|---|---|---|---|---|---|---|
| Oligo- | % of administered radioactivity recovered per gram of tissue | | | | | |
| nucleotide | Spleen | Liver | Brain | Kidney | Pancreas | Blood |
| 10-mer | 1.127 ± 0.13 | 0.37 ± 0.028 | 0.0074 ± 0.0020 | 0.289 ± 0.026 | 0.079 ± 0.042 | 0.18 ± 0.014 |
| 42-mer | 0.84 ± 0.095 | 0.313 ± 0.052 | 0.0075 ± 0.0034 | 0.6 ± 0.052 | 0.194 ± 0.04 | 0.11 ± 0.034 |

Nos. calculated as percent of radioactive oligonucleotide administered/gm of the tissue.

Distribution of radioactivity of the 10- and 42-mers is shown in Table 2. Interestingly, by oral administration, some of the oligonucleotide crossed the blood brain barrier and noticeable amount of oligonucleotides were recovered in the brain, which significantly increased from 1 hour to 4 hours. The pattern of distribution of antisense oligonucleotides did not change with the size of the oligonucleotide. Four hours after administration, the distribution of labeled oligonucleotide had undergone noticeable changes compared to 1 hr. In the case of short oligonucleotide, major percent of radioactivity was in liver 0.79±0.096%. This was followed by 0.67±0.062 in kidney, spleen (0.25±0.056%) and pancreas (0.104±0.003%).

TABLE 2
Distribution and recovery of antisense oligonucleotides in various tissues 4 hours after administration.

| Size of | | | | | | |
|---|---|---|---|---|---|---|
| Oligo- | % of administered radioactivity recovered per gram of tissue | | | | | |
| nucleotide | Spleen | Liver | Brain | Kidney | Pancreas | Blood |
| 10-mer | 0.25 ± 0.056 | 0.79 ± 0.096 | 0.053 ± 0.0058 | 0.67 ± 0.062 | 0.104 ± 0.0037 | 0.052 ± 0.0053 |
| 42-mer | 0.616 ± 0.062 | 1.14 ± 0.37 | 0.044 ± .0.0066 | 0.738 ± 0.15 | 0.214 ± 0.078 | 0.0783 ± 0.014 |

Nos. calculated as percent of radioactive oligonucleotide administered/gm of the tissue.

Long oligonucleotide essentially followed similar pattern. However, the amount of oligonucleotide recovered in the spleen was reduced by approximately 50% of that recovered from liver four hours after administration when compared to the distribution at one hour. The amount recovered in the brain increased three fold from 1 hour to four hours. The amount recovered from kidney has also increased by almost 45% while the amount recovered in pancreas showed a decrease.

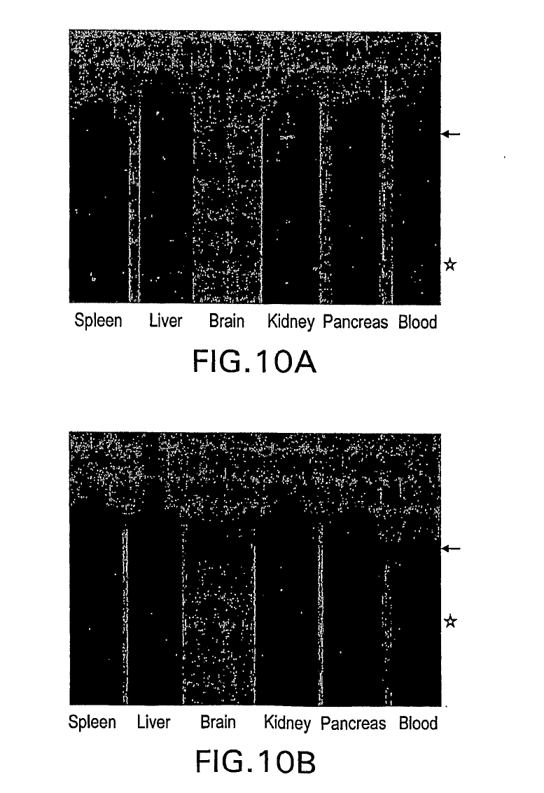

STABILITY OF THE TRANSPORTED OLIGONUCLEOTIDES: In order to study the stability of the oligonucleotide transported, the tissues were homogenized and the soluble fractions were subjected to polyacrylamide gel electrophoresis. The oligonucleotide could be detected in all the tissues tested. The transport into various tissues could be observed for both short and long oligonucleotides. Extensive degradation of oligonucleotides was observed in all tissues except in the brain. No visible band of oligonucleotides was observed in the brain one hour after administration although, all other tissues showed a distinct bands of radioactivity oligonucleotides. However, four hours after administration, a clear band of oligonucleotide could be detected in the brain tissue also (FIG. 10). No degradation was seen in the brain or other tissues at the latest time point measured after i.v. injection, which was 16 hrs.

Radioactivity had a retarded migration in the gel compared to free oligonucleotide. The arrow in the figure shows the position of major band of radioactivity. The star represents the position of free oligonucleotide. This suggests that the oligonucleotides may be associated with a binding element, possibly a transporter. The pattern of distribution of oligonucleotide is the same for 1 hour and 4 hours. There fore, the figure for 1 hour is not given to avoid repetition.

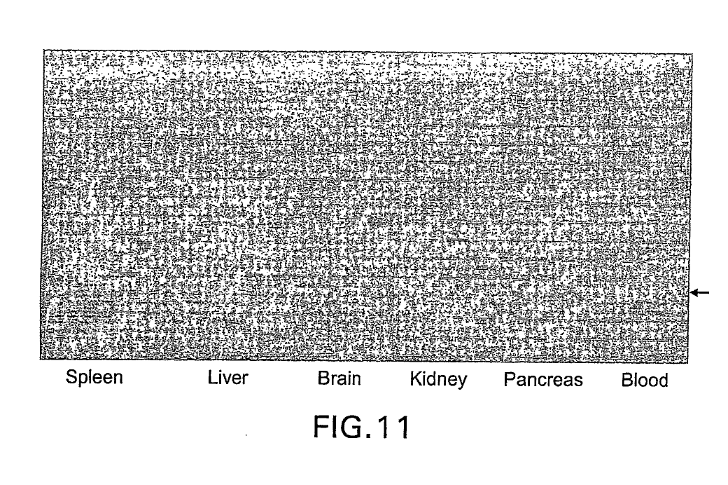

Upon phenol:chloroform extraction of the tissue homogenates, the radioactivity migrated as free oligonucleotide, confirming the association of antisense oligonucleotides with cellular component(s) (FIG. 11). The figure shows the extraction of 42-mer from the tissues.

Several studies have widely proposed the use of antisense oligonucleotides as potential therapeutic agents due to their specificity in blocking the transcriptional ability of targeted messages (Chiang et al., 1991; Woolf et al., 1992). Although antisense oligonucleotides are potent gene regulators (Flanagan and Wagner, 1997; Wagner, 1994), their stability and efficiency of transport limit their usage as therapeutic agents. In order to delay their degradation, various modifications to the nucleotides have been used (Wagner et al., 1993; Gutierrez et al., 1997; Flanagan et al, 1996). The inventors and others have reported APP inhibition by antisense oligonucleotides in tissue culture cells (Coulson et al., 1997). The rate-limiting factor in cell suspensions is the efficiency of transport into cells. Various methods have been used to increase the efficiency of transport in tissue culture cells (Kumar et al., 1974; Garcia-Chaumont et al., 2000; Garcia-Chaumont et al., 2000). These involve encapsulation of the oligonucleotide by cationic agents or mechanical disruption of cells in such a way that temporary pore formation occurs allowing passive transport. However, the inventors have shown that antisense oligonucleotides raised against APP can act in vivo. Either antisense delivered by i.c.v (Kumar et al, 2000) or i.v. injection had similar effects (Banks et al, 2001). This suggests that the transport of oligonucleotides in vivo may be facilitated by as yet unrecognized factors.

In vivo the efficiency of antisense oligonucleotide is limited by the amount of oligonucleotide taken up by the targeted tissue. Therefore, analysis of tissue distribution of the administered oligonucleotide will be a valuable tool to evaluate possible side effects and the amount of oligonucleotide needed to inhibit gene expression in the target tissue. In the case of proteins like APP, which deposit amyloid plaques by abnormal processing in neurodegenerative disorders like Alzheimer's disease, the targeted antisense has to reach the brain. In order to assess the extent of transport into the brain, the inventors studied the tissue distribution and the stability of antisense oligonucleotides in a mouse system for this study.

Labeled antisense oligonucleotides could be detected in the brain tissues in one hour after oral administration. The extent of transport increased by three-fold in 4 hours The inventors have already shown that oligonucleotides injected into the blood are transported into the brain (Banks et al., 2001). Therefore it was not surprising that the amount of oligonucleotide recovered in the brain increased with time. Further, intact oligonucleotide could be recovered from all tissues tested although some of the administered oligonucleotide was degraded. The fact that the undegraded oligonucleotides exhibited retarded mobility in the polyacrylamide gels compared to the free oligonucleotide suggests that binding to proteins, possibly the transporter proteins, is occurring. The association of oligonucleotide to an 80 kDa protein (Loke et al., 1989) and a 30 kDa protein (Bennett and Stephenson, 1978) in a cell culture system has been reported.

Results here show that oligonucleotides may be taken up after oral administration with distribution into various organs. Oral administration results in the transport of 1-2% of the antisense oligonucleotide into the brain. Oligonucleotide in brain appears to be more stable than the when transported into other organs, as the oligonucleotide band found in the brain showed no degradation products (even in other tissues, no degradation was seen 16 hrs after i.v. injection). Further, in the studies presented in this investigation, the inventors have used end-labeled oligonucleotides. The end-labeled phosphate is more vulnerable to detachment than internally labeled one. Therefore, the amount of intact oligonucleotide may be more than that observed in these studies if the oligonucleotide is 2-O-methylated or propylated at the same time improving their efficiency of inhibition of the targeted mRNA (Chauhan, 2002; McKay et al., 1996). In addition, proteins like APP are ubiquitously distributed in all tissues in vertebrates. APP is known to be functionally important in tissues for endocytosis and phagocytosis and may play a key role in structural integrity of the cell (Beer et al., 1995; Hung et al., 1996; Nordstedt et al., 1994). Therefore, any antisense sequence directed against APP should be tissue specific in order to reduce any deleterious to other organs. Thus, the usage of antisense oligonucleotides against APP by oral administration must be taken with caution. While the oral administration of antisense oligonucleotides to specific targets may have therapeutic value, its wide tissue distribution and probable extensive degradation must be kept in mind before designing the oligonucleotides.

### REFERENCES

The following references, to the extent that they provide exemplary procedural or other details supplementary to those set forth herein, are specifically incorporated herein by reference.
U.S. Pat. No. 5,304,506
U.S. Pat. No. 5,489,677
U.S. Pat. No. 5,539,082
U.S. Pat. No. 5,602,240
U.S. Pat. No. 5,714,331
U.S. Pat. No. 5,719,262
U.S. Pat. No. 6,310,048
U.S. Pat. No. 6,310,048
Banks et al., J. Pharm. Exper. Therap., 297(3):1113-1121, 2001.
Beer et al., Neurodegeneration, 4(1):51-59, 1995.
Bennet et al., J. Clinical Invest., 76:2182-2190, 1985.
Bennett et al., J. Immunology, 152(7):3530-3540, 1994.
Chauhan, Antisense Res. Drug Dev., 12(5):353-357, 2002.
Chiang et al. J. Biolog. Chem., 266(27):18162-18171, 1991.
Cleary et al., Brain Res., 682:69-74, 1995.
Coulson et al., Brain Res., 770(1-2):72-80, 1997.
Crooke, Antisense Res. Drug Dev., 3(1):1-2, 1993.
Dawson et al., Neurosci., 90:1-13, 1999.
Dolnick, Cancer Invest., 9(2):185-194, 1991.
Flanagan and Wagner, Molec. Cellular Biochem., 172(1-2):213-225, 1997.
Flanagan et al., Nature Biotechnol., 14(9):1139-1145, 1996.
Flood et al., Brain Res., 663:271-176, 1994b.
Flood et al., Proc. Natl. Acad. Sci. USA, 88:3363-3366, 1991.
Flood et al., Proc. Natl. Acad. Sci. USA, 91:380-384, 1994a.
Garcia-Chaumont et al., Antisense Res. Drug Dev., 10(3):177-184, 2000.
Garcia-Chaumont et al., Pharmacol Therap., 87(2-3):255-277, 2000.
Gutierrez et al., Biochemistry, 36(4):743-748, 1997.
Hardy and Selkoe, Science, 297:353-356, 2002.
Hastie and Held, Proc. Natl. Acad. Sci. USA, 75:1217-1221, 1978.
Huber et al., Brain Res., 603:348-352, 1993.
Itoh et al., Eur. J. Pharmacol., 382:167-175, 1999.
Johansson et al., Nucleic Acids Res., 22(22):4591-4598, 1994.
Jung et al., J. Neurosci. Res., 46(3):336-348, 1996.
Kim et al., J. Neurosci., 21:1327-1333, 2001.
Kumar et al., Nature, 250(464):323-325, 1974.
Kumar et al., Peptides, 21(12):1769-1775, 2000.
Loke et al., Proc. Natl. Acad. Sci. USA, 86:3474-3478, 1989.
McKay et al., Nucleic Acids Res., 24(3):411-417, 1996.
Mileusnic et al., Eur. J. Neurosci., 12:4487-4495, 2000.
Milligan et al., J. Med. Chem., 36(14):1923-1937, 1993.
Morley et al., Neurobiol. Learning Mem., 78:125-138, 2002.
Morton et al., Neurosci. Lett., 319:37-40, 2002.
Nielsen et al., Science, 254:1497-1500, 1991.
Nordstedt et al., J. Biolog. Chem., 269(13):9805-9810, 1994.
Paterson et al., Proc. Natl. Acad. Sci. USA, 74:4370-4374, 1977.
PCT Appln. WO 01/42266,
Raymond et al., Brain Res., 968:263-272, 2003.
Robinson and Bishop, Neurobiol. Aging, 23:1051-1072, 2002.
Sambrook et al., In: Molecular cloning: a laboratory manual, 2^{nd} Ed., Cold Spring Harbor Laboratory Press, Cold Spring Harbor, N.Y., 1989.
Seabrook et al., Neuropharmacology, 38:349-359, 1999.
Terranova et al., Neurosci Letters, 213:79-82, 1996.
Trubetskaya et al., Neurosci. Behav. Physiol., 33:95-98, 2003.
Wagner et al., Science, 260(5113):1510-1513, 1993
Wagner, Nature, 372(6504):333-335, 1994.
Walder et al., Proc. Natl. Acad. Sci. USA, 85:5011-5015, 1988.
Woolf et al., Proc. Natl. Acad. Sci. USA, 89:7305-7309, 1992.
Wu et al., Eur. J. Pharmacol., 284:R1-3, 1995.
Zamecnik and Stephenson, Proc. Natl. Acad. Sci. USA, 75:280-284, 1978.

1. A method of improving learning and/or memory, the method comprising administering to the patient an amyloid precursor protein (APP) modifying amount of a compound.

2. The method as set forth in claim 1, wherein the compound administered increases the level of APP.

3. The method as set forth in claim 1, wherein the compound administered decreases the level of APP.

4. The method as set forth in claim 1, wherein the patient is a mammal.

5. The method as set forth in claim 4, wherein the patient is a human.

6. A method of improving learning and/or memory, the method comprising administering to the patient a compound that optimizes the levels of amyloid precursor protein (APP) and/or beta amyloid (Aβ) protein.

7. A method of improving cognitive function, the method comprising administering to a subject a therapeutically effective amount of an amyloid precursor protein (APP) and/or beta amyloid (Aβ) protein inhibiting compound.

8. The method as set forth in claim 7, wherein the compound blocks the amino acid sites 18-22 of the Aβ protein.

9. A method of improving learning and/or memory, the method comprising administering to the patient a therapeutically effective amount of a compound that increases the amount of amyloid precursor protein (APP) and/or beta amyloid (Aβ) protein.

10. The compound as set forth in claim 9, wherein the compound is Aβ 1-42.

11. The compound as set forth in claim 9, wherein the compound is Aβ 12-28.

12. A method of treating a disease or condition in a patient, where the disease is associated with the expression of amyloid beta (Aβ) protein, the method comprising administering to the patient a therapeutically effective amount of at lease one amyloid precursor protein (APP) regulating compound.

13. The method as set forth in claim 12, wherein the disease or condition is memory loss.

14. The method as set forth in claim 12, wherein the disease or condition is Alzheimers disease.

15. A pro-memory drug compound which regulates the levels of amyloid precursor protein (APP) and/or beta amyloid (Aβ) protein.

16. The compound set forth in claim 15, wherein the compound is selected from the group consisting of Aβ antibody, Aβ protein, antisense polynucleotides, Aβ 1-42, and Aβ 12-28.

17. The compound as set forth in claim 15, wherein the compound is Aβ 1-42.

18. The compound as set forth in claim 15, wherein the compound is Aβ 12-28.

19. The compound as set forth in claim 15, wherein the APP/Aβ levels are increased.

20. The compound as set forth in claim 15, wherein the APP/Aβ levels are decreased.
